(19) United States
(12) Patent Application Publication
(10) Pub. No.: US 2025/0288403 A1
(43) Pub. Date: Sep. 18, 2025

(54) PRE-SHAPED ALLOGRAFT IMPLANT FOR PELVIC ORGAN PROLAPSE

(71) Applicant: ALLOSOURCE, Centennial, CO (US)

(72) Inventors: McKenzie Pietrafeso, Denver, CO (US); Charles Manuele, Aurora, CO (US); Madeline Dahl, Castle Rock, CO (US); Reginald Stilwell, Parker, CO (US)

(21) Appl. No.: 19/079,863

(22) Filed: Mar. 14, 2025

Related U.S. Application Data
(60) Provisional application No. 63/565,294, filed on Mar. 14, 2024.

(51) Int. Cl. A61F 2/00

(52) U.S. Cl. CPC A61F 2/0063; A61F 2/0095; A61F 2002/0068

(57) ABSTRACT

An acellular dermal matrix (ADM) graft product for a pelvic organ prolapse repair procedure includes an ADM graft derived from full-thickness skin, with a pre-formed shape having one or more of a mesh pattern formed in a central portion thereof, suture punch or slit features or radii transitions about the perimeter. The ADM graft is pre-hydrated, placed in a sterilization package and irradiated to provide a two-year shelf-life for the ADM graft product, which is used in the time-saving, efficient, and effective surgical reconstruction of soft tissue defects. One manufacturing method involves providing a portion of full-thickness donor-derived skin, removing an epidermis, basement membrane, and a fat layer form the portion of the skin, decellularizing the skin to form a portion of ADM graft material, pre-shaping and meshing the ADM graft material, verifying a thickness of the ADM graft material, and packaging the pre-shaped, meshed ADM graft material in a sterilizable package and irradiating for a two-year shelf-life to form the ADM graft product. Other embodiments are also disclosed.

### REFERENCE TO PENDING PRIOR PATENT APPLICATION

This application claims the benefit under 35 U.S.C. 119 (e) of U.S. Provisional Patent Application No. 63/565,294, filed Mar. 14, 2024 by McKenzie Pietrafeso for “PRE-SHAPED ALLGRAFT IMPLANT FOR PELVIC ORGAN PROLAPSE” which patent application is hereby incorporated herein by reference.

### BACKGROUND

An allograft includes bone, tendon, skin, or other types of tissue that is transplanted from one person to another. Allografts are used in a variety of medical treatments, such as knee replacements, bone grafts, spinal fusions, eye surgery, and skin grafts for reconstructive surgery and for the severely burned. Allografts come from voluntarily donated human tissue obtained from cadaveric donor-derived, living-related, or living-unrelated donors and can help patients regain mobility, restore function, enjoy a better quality of life, and even save lives in the case of cardiovascular tissue or skin.

An acellular dermal matrix (ADM) graft is a soft tissue graft generated by a decellularization process that preserves the intact extracellular skin matrix. Upon implantation, the ADM structure serves as a scaffold for donor-side cells to facilitate subsequent incorporation and revascularization. ADMs are manufactured utilizing known methods of decellularization by means of ionic and nonionic detergent methods, as well as those utilizing enzymatic processes and other techniques such as those listed in “Decellularization of Tissues and Organs,” Gilbert, et al, 2006 (https://www.ncbi.nlm.nih.gov/pubmed/16519932).

Currently, ADM grafts are primarily derived from decellularized cadaveric skin and must be shaped and/or cut as necessary by the surgeon either prior to or during a surgical procedure. Such grafts are also commonly formed from solid or perforated ADM. As a result, existing ADM grafts present efficiency, efficacy, and repeatability challenges when used for reconstructive surgery purposes.

Problems exist seeking solutions for treating pelvic organ prolapse, urinary incontinence, fecal incontinence, urethral diverticulum, fistula, neo-vagina, and wound healing abnormalities.

Naturally occurring extracellular matrices (ECMs), known as autografts, have been used for tissue repair and regeneration, including usable for repair, support and stabilization of anatomical defects. There are known techniques for harvesting and preparing ECMs ahead of surgery, including sterilization and related techniques for minimizing post-surgical complications.

One common problem arises due to the nature of these grafts having a general non-defined shape. For certain types of surgeries, the graft must be cut into a specific shape or design. Because of sterilization requirements for surgical theaters, having to cut the graft during surgery adds additional complications, including causing the surgery to become longer in duration, lack of precision with available tools in an operating room, lack of knowledge by the surgeon and/or staff to create an appropriate design, as well as adding contamination concerns.

Therefore, there exists a need for a biologic vaginal graft providing for improving not only the patient outcome but also improving the safety and timing of any attendant surgeries.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides a method of manufacturing an acellular dermal matrix (ADM) graft product for use in a reconstructive surgical procedure. The method may include the following steps: (1) providing a portion of donor-derived skin, the portion of the donor-derived skin having a full thickness; (2) removing an epidermis and basement membrane layer and a fat layer from the portion of the donor-derived skin to form a portion of dermal tissue; (3) decellularizing the portion of the dermal tissue to form a portion of ADM graft material; (4) forming the portion of the ADM graft material into a pre-defined shape in anticipation of the reconstructive surgical procedure; (5) fenestrating the pre-defined shape into a mesh pattern; (6) verifying that a thickness of the pre-defined shape equals a specified thickness; (7) packaging the pre-defined shape in a medical sterilization pouch to form a packaged, pre-shaped, and meshed ADM graft; and (8) irradiating the packaged, pre-shaped, and meshed ADM graft to a sterility assurance level of 10⁻⁶ to form the ADM graft product.

Another embodiment provides a pre-shaped, meshed acellular dermal matrix (ADM) graft stored as a packaged graft product prepared by a process comprising the steps of: (1) providing a portion of ADM tissue having a thickness between 1 mm and 2 mm; (2) fenestrating the portion of the ADM tissue in a mesh pattern extending over an entirety of the portion of the ADM tissue; (3) scoring the portion of the ADM tissue into a pre-defined shape to form the pre-shaped, meshed ADM graft; (4) verifying the thickness of the pre-shaped, meshed ADM graft; (5) packaging the pre-shaped, meshed ADM graft in a medical sterilization pouch; and (6) irradiating the pre-shaped, meshed ADM graft within the medical sterilization pouch to a sterility assurance level of 10⁻⁶ to form the packaged graft product.

Yet another embodiment provides an acellular dermal matrix (ADM) graft product. The ADM graft product may include an ADM graft derived from full-thickness skin, the ADM graft having a pre-formed shape with a mesh pattern formed therein, as well as a medical sterilization pouch sealed about the ADM graft, wherein when the medical sterilization pouch and the ADM graft are irradiated to a sterility assurance level of 10⁻⁶, the ADM graft product has a shelf-life of two years.

Other embodiments provide an ADM graft that combines the ADM as designed with antimicrobial elements that mitigate or prevent complications arising from post-surgical infections. Antimicrobial agents that are compatible with the ADM include silver in its colloidal, elemental or ionic form. The silver may be complexed with chelating agents or may be added directly to the ADM prior to final packaging. Similarly other antimicrobial agents may be combined with the ADM. Other agents well known to be used medically are chlorhexidine gluconate and antimicrobial peptides of various amino acid chain length.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 illustrates a pelvic organ prolapse repair implant for an anterior approach procedure of one embodiment of a pre-shaped, partially meshed acellular dermal matrix (ADM) graft derived from full-thickness skin;

FIG. 2 illustrates a pelvic organ prolapse repair implant for a posterior approach procedure of one embodiment of a pre-shaped, partially meshed acellular dermal matrix (ADM) graft derived from full-thickness skin;

FIGS. 3-8 illustrate various exemplary mesh patterns which may be configured in the central region of the ADM graft;

FIG. 9 illustrates a pelvic organ prolapse repair implant for an anterior approach procedure of one embodiment of a pre-shaped, partially meshed acellular dermal matrix (ADM) graft derived from full-thickness skin; and

FIG. 10 illustrates a pelvic organ prolapse repair implant for a posterior approach procedure of one embodiment of a pre-shaped, partially meshed acellular dermal matrix (ADM) graft derived from full-thickness skin.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

The present disclosure includes a biological vaginal graft with a central body and a plurality of extension members. The central body has a tapered dimension from a bottom portion to a top portion. The extension members extend outward from the central body, disposed on opposing sides of the bottom portion. The central body and the extension members are composed of tissue graft material. The extension members, as being composed of the tissue graft material, are affixable via a grafting medical procedure to a vaginal canal of a patient.

In varying embodiments, the biological vaginal graft is composed of different biologically-compatible material. One embodiment includes composition of decellularized dermal allograft. Another embodiment includes composition of animal-based origin. Additional embodiments include composition of other human tissue such as fascia lata, amnion, other dermal tissues or other biologically compatible human tissues.

One embodiment provides a method of manufacturing an acellular dermal matrix (ADM) graft product for use in a reconstructive surgical procedure. The method may include the following steps: (1) providing a portion of donor-derived skin, the portion of the donor-derived skin having a full thickness; (2) removing an epidermis and basement membrane layer and a fat layer from the portion of the donor-derived skin to form a portion of dermal tissue; (3) decellularizing the portion of the dermal tissue to form a portion of ADM graft material; (4) forming the portion of the ADM graft material into a pre-defined shape in anticipation of the reconstructive surgical procedure; (5) fenestrating the pre-defined shape into a mesh pattern; (6) verifying that a thickness of the pre-defined shape equals a specified thickness; (7) packaging the pre-defined shape in a medical sterilization pouch to form a packaged, pre-shaped, and meshed ADM graft; and (8) irradiating the packaged, pre-shaped, and meshed ADM graft to a sterility assurance level of 10⁻⁶ to form the ADM graft product.

Another embodiment provides a pre-shaped, meshed acellular dermal matrix (ADM) graft stored as a packaged graft product prepared by a process comprising the steps of: (1) providing a portion of ADM tissue having a thickness between 1 mm and 2 mm; (2) fenestrating the portion of the ADM tissue in a mesh pattern extending over an entirety of the portion of the ADM tissue; (3) scoring the portion of the ADM tissue into a pre-defined shape to form the pre-shaped, meshed ADM graft; (4) verifying the thickness of the pre-shaped, meshed ADM graft; (5) packaging the pre-shaped, meshed ADM graft in a medical sterilization pouch; and (6) irradiating the pre-shaped, meshed ADM graft within the medical sterilization pouch to a sterility assurance level of 10⁻⁶ to form the packaged graft product.

Yet another embodiment provides an acellular dermal matrix (ADM) graft product. The ADM graft product may include an ADM graft derived from full-thickness skin, the ADM graft having a pre-formed shape with a mesh pattern formed therein, as well as a medical sterilization pouch sealed about the ADM graft, wherein when the medical sterilization pouch and the ADM graft are irradiated to a sterility assurance level of 10⁻⁶, the ADM graft product has a shelf-life of two years.

Other embodiments provide an ADM graft that combines the ADM as designed with antimicrobial elements that mitigate or prevent complications arising from post-surgical infections. Antimicrobial agents that are compatible with the ADM include silver in its colloidal, elemental or ionic form. The silver may be complexed with chelating agents or may be added directly to the ADM prior to final packaging. Similarly other antimicrobial agents may be combined with the ADM. Other agents well known to be used medically are chlorhexidine gluconate and antimicrobial peptides of various amino acid chain length.

Various embodiments of the products and associated methods of manufacture and use described herein relate to a pre-shaped, meshed or fenestrated acellular dermal matrix (ADM) graft derived from full-thickness human, donor-derived skin for use in the surgical reconstruction of soft tissue defects resulting from trauma, disease, or surgical intervention. For example, embodiments of the ADM graft discussed herein may be used in the surgical specialty of plastic surgery, and particularly in prepectoral and post-mastectomy breast reconstruction, where the ADM graft is an adjunct to integumental repair of the surgical site.

Embodiments of the ADM graft may be packaged and irradiated for long-term sterile storage in a manner that allows them to be used in surgical procedures within two years of packaging. The ADM graft is packaged moist, in sterile water and is immediately ready to use out of sterile packaging. In various embodiments, the graft is pre-hydrated in packaging with about 2-5 mL of sterile water. In use, embodiments of the pre-shaped, meshed ADM graft provide the surgeon with a mechanism to restore function to and support integumental tissue after surgical intervention in a manner that is repeatable, effective, and time efficient by leveraging a manufactured, pre-shaped and meshed ADM graft product that is derived from full-thickness skin. Embodiments of the ADM graft facilitate fluid drainage from the surgical site to discourage seroma formation, increase the rate of integration of the ADM graft with the body, and provide a reliable, repeatable solution the surgeon may use “off the shelf” rather than utilizing valuable time and resources for graft processing in preparation for or during the surgical procedure.

In the present disclosure, there is provided an allograft solution for pelvic organ prolapse (POP). Acellular dermal matrix allografts are commonly used in this procedure, but the rectangular grafts need to be cut to the proper shape in the operating room. This development is a ready-to-use allograft and contains features to improve the performance of the graft.

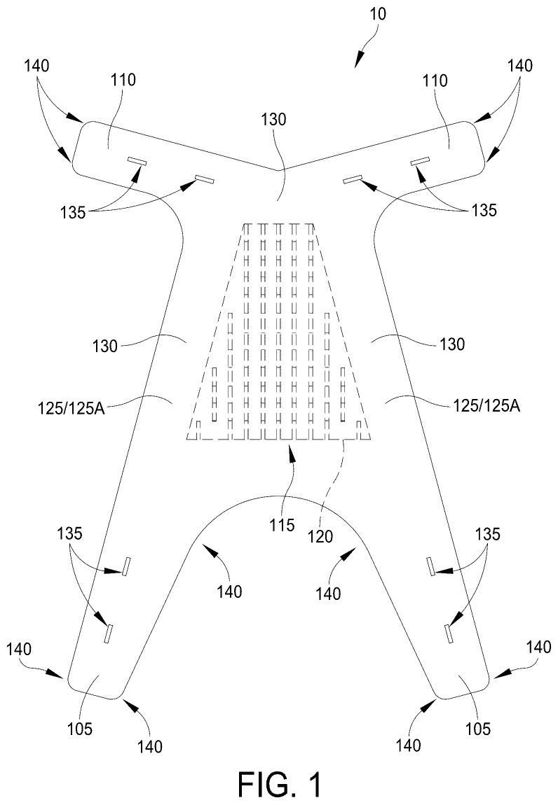

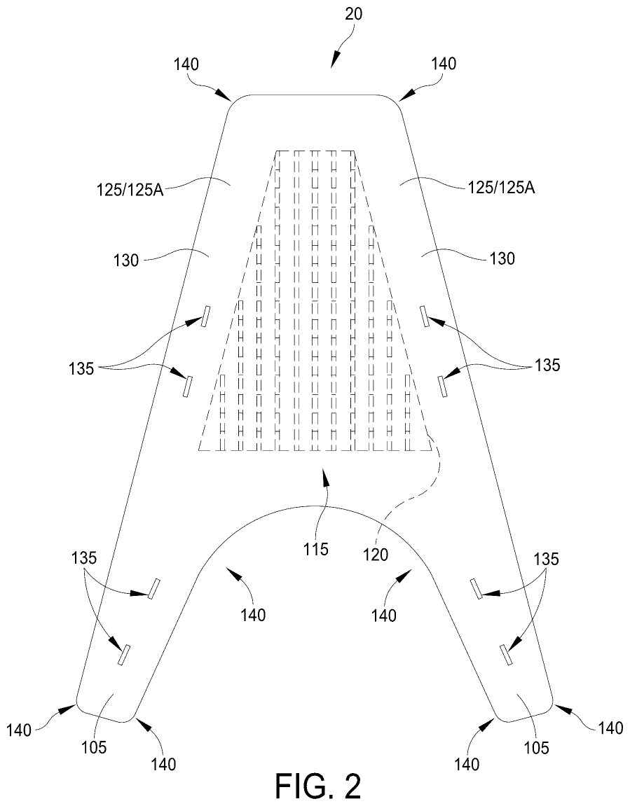

With reference to FIGS. 1 and 2, and in various embodiments, there are shown POP implants 10, 20 of a pre-shaped, meshed ADM graft derived from decellularized, full-thickness skin. Using full-thickness skin as the source for the ADM graft ensures that the ADM graft has sufficient biomechanical properties to support varying surgical requirements, including, for example, a suitable ultimate tensile strength, suture pull-out resistance, and a Young's modulus indicative of a soft and supple graft.

In these various embodiments, the pre-shaped, meshed ADM graft 10, 20 may have a pre-formed shape to conform to the female pelvic anatomy. A set of lower strips 105 and a set of upper strips 110 may be provided. The lower strips 105 allow for anchoring to pelvic ligaments while the central portion 115 supports the vaginal mucosa tissue. The central portion 115 is meshed 120 to allow for graft stretching and for fluid drainage. Additionally, the intercedes increase the surface area for incorporating into surrounding vascular tissue. Strategically placed slits may be provided in the non-meshed regions (e.g., the perimeter margin 125) through which the surgeon may place sutures to anchor the graft. In one embodiment, this central meshed area 115 provides a suture border region 130, which in turn contains suture punch points 135 for quick and optimal placement of suture by a surgeon, and the perimeter of the implant is configured with a radii of transition areas 140 so as to provide enhanced resistance to stress tears.

In general, there are two embodiments of similar overall configurations. A first design, as shown in FIG. 1, has strips 105, 110 on the top and bottom, respectively. This first configuration is referred to as a pelvic organ prolapse repair implant for an anterior approach procedure. A second configuration, as shown in FIG. 2 has strips 105 only on the bottom. This second configuration is referred to as a pelvic organ prolapse repair implant for a posterior approach procedure. These two general configurations allow for two different surgical approaches.

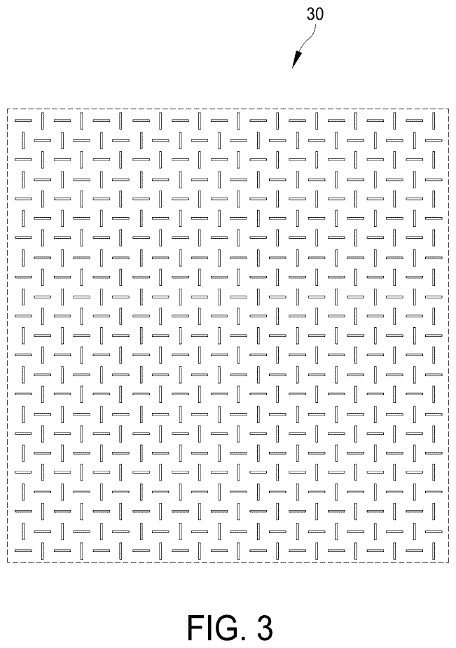

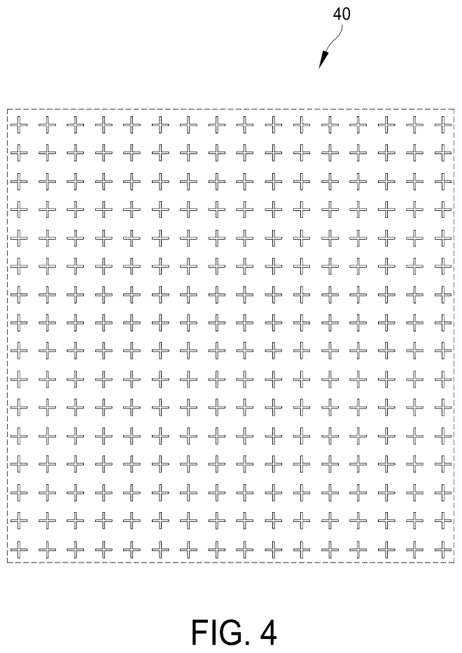

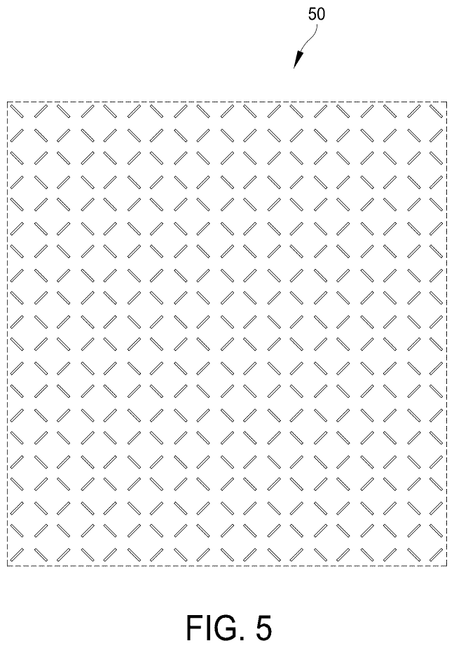

Both of these general configurations, the anterior design and the posterior design, may have various features. One feature is a central meshed region 115, 120 with unmeshed suturing portions 125A around the perimeter 125 of the implant. In one embodiment, the mesh 120 may run parallel to the long axis of the graft (in which the long axis is defined as running between the posterior and anterior ends of the implant. Mesh 120 may include various types of uniform mesh or non-uniform mesh. Examples of unform mesh patterns 30-80 are provided in FIGS. 3-8.

Both of these configurations may also have slits 130 or suture punch points 130 in the non-meshed regions 125A (e.g., the unmeshed suturing portions 125A) to allow for graft suturing without needing to locate a suitable suture location and drive a suture hole in the surgical theater.

Both of designs may be fabricated with deep reticular acellular dermal matrix, which allows for better incorporation into the human body. This deep reticular acellular dermal matrix may be about 1.0-2.0 mm thick.

Alternative embodiments of these general configurations may include different overall sizes, thicknesses, different angles of the strips, different widths of the strips, with different meshing ratios, etc. It may be made from human ADM which contains the basement membrane. It may also include acellular dermal matrices originating from animal sources including porcine, bovine or other xenograft sources.

Additionally, any form of the implants of this present disclosure made from ADM may be impregnated with an antimicrobial agent such as minocycline, rifampin, gentamycin, vancomycin, or any number of antibiotic or antimicrobial agents to reduce the possibility of post-surgical infections. Post-surgical infection is a distinct possibility after pelvic surgical procedures and inclusion of a pre-impregnated ADM will minimize the chance of infection.

In at least one embodiment, the perimeter 125 of the pelvic organ prolapse repair implant may include a significantly curved radii 140 at transition portions. These radii 140 provide functionality which includes decreased incidence for a breaking point as well as on the interior corners will avoid stress concentration centration and associated tearing and the external corners will also avoid tearing.

Many other allograft solutions on the market are either rectangular and need to be cut to shape by the surgeon in the operating room. Some allograft solutions are of a somewhat similar shape, but do not contain any of the specified meshing placement or suture slit features or radii transitions about the perimeter. These features are provided by the present disclosure to improve performance of the graft implant and improve ease of use by the surgeon.

Without these features, other allograft implant devices are generally deficient as lacking fluid drainage or stretching in the central region of the currently used allograft implants. These currently used allograft implants lack any indicia and provision of secure locations for the surgeon to place sutures to anchor the allograft in place. Additionally, other alternatives typically come dehydrated and require hydration prior to use.

In various embodiments, the allograft implants of the present disclosure are formed of deep reticular human acellular dermal matrix (ADM). The ADM is split along its thickness into an upper and lower portion. The lower portion is cut to shape and meshed using a cutting die. It is then packaged flat with sterile gauze backing in a sterile plastic sleeve with sterile water, and then electron beam irradiated to SAL 10-6.

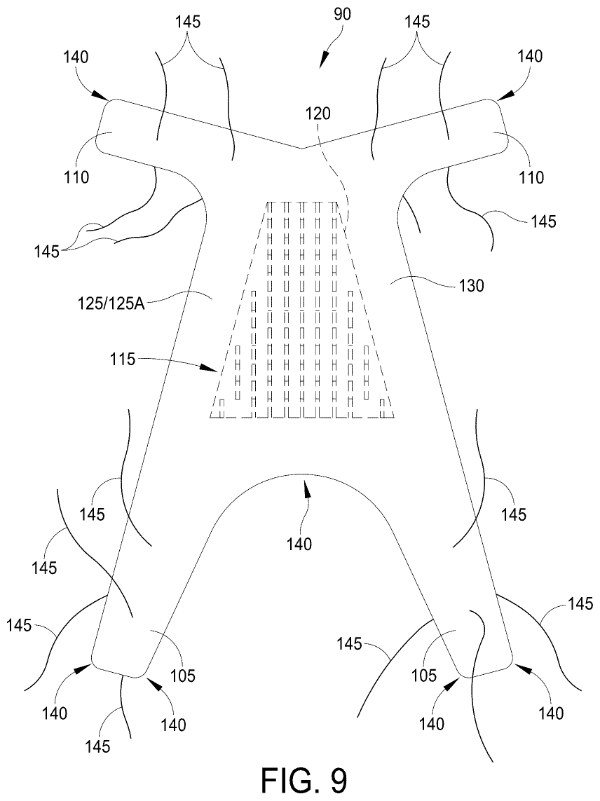

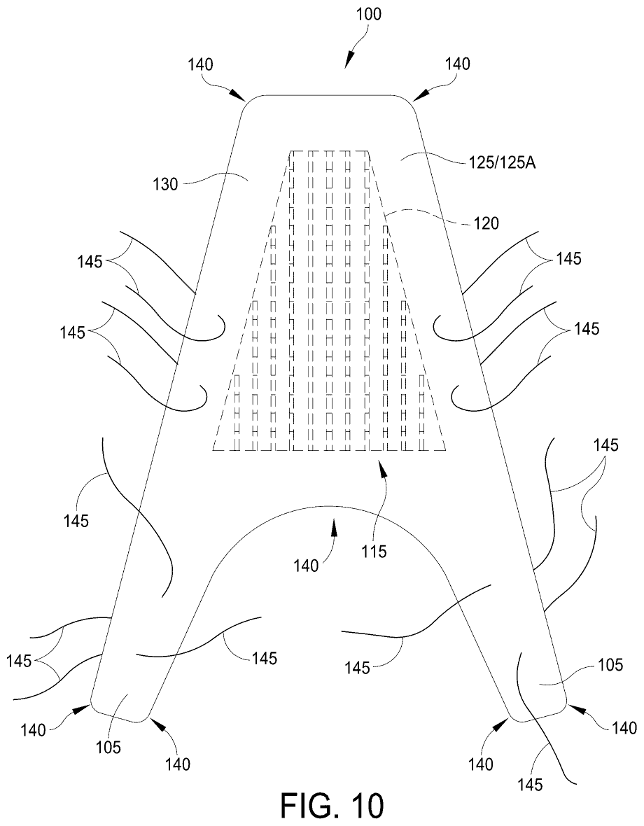

With reference now to FIGS. 9 and 10, in yet another embodiment, the above-disclosed allograft implants may be provided with pre-incorporated sutures 145. The sutures may be bioresorbable or permanent materials. Bioresorbable sutures, if pre-incorporated, would allow for the slow healing response of the pelvic region to occur while the suture slowly resorbs.

In still other embodiments, the above-disclosed allograft implants may be provided in a configuration as a urinary incontinence sling.

In various manufacturing processes may be used to produce one or more of the above-identified allograft implants. In various embodiments, decellularized, full-thickness dermal tissue may be shaped and cut into the pre-shaped ADM graft using an appropriately designed scoring tool along with a cutting tool such as, for example, a surgical scalpel or a surgical scissor.

The pre-shaped nature of the ADM graft disclosed herein saves the surgeon valuable time during a surgical procedure because there is no (or minimal) need for the surgeon to shape, cut, or otherwise form the ADM graft into a desired shape during surgical preparation. Instead, the surgeon may simply select an appropriately pre-shaped ADM graft for the particular surgery and proceed.

Embodiments of the pre-shaped ADM graft may additionally include a mesh or fenestration pattern to allow for increased fluid flow through the graft, thereby reducing the chances of post-surgical seroma formation, a frequent complication after surgeries using existing ADM grafts. Pre-meshing also prevents the surgeon from having to perform any type or kind of meshing procedures during surgical preparation or during a surgical procedure and ensures an optimal mesh ratio to provide maximum fluid egress, or drainage, from the surgical site to prevent seroma formation and a maximum graft surface area for improved integration into the body post procedure.

FIGS. 3-8 provides exemplary meshing options 30-80. The mesh or fenestration pattern may be formed in the pre-shaped, meshed ADM graft using a 60-ton die-cut press to stamp out the fenestration pattern and may be configured to allow for only portions of the graft to be meshed.

In one embodiment, the pre-shaped, meshed ADM graft 10, 20 may be formed of the ADM derived from full-thickness skin, as discussed above, combined with antimicrobial elements that mitigate or prevent complications arising from post-surgical infections. Antimicrobial agents compatible with the ADM may include, for example, silver in its colloidal, elemental, or ionic form. The silver may be complexed with chelating agents or may be added directly to the ADM prior to final packaging. Similarly, other antimicrobial agents may be combined with the ADM. Other agents known to be used medically may include chlorhexidine gluconate and antimicrobial peptides having various amino acid chain lengths.

After manufacture and to provide completely hydrated allograft, packaged moist in sterile water, a shelf-stable, packaged ADM graft product, the pre-shaped, meshed ADM graft may be packaged along with two opposing pieces of backing material and sterile water in a sealed medical sterilization pouch such as, for example, a Kapak pouch (manufactured by AMPAK Technology Inc. of Larchmont, NY), or further into a sealed, peelable medical sterilization pouch known as a “peel pouch” or a “chevron pouch.” The packaged ADM graft product may then be irradiated to a sterility assurance level (SAL) of 10⁻⁶ such that it may be stored at room temperature for up to two years. The packaged ADM graft product may be labeled in any appropriate manner and may include information pertaining to the raw material, the shape, a use by date, special requirements, results of a visual inspection, and so on.

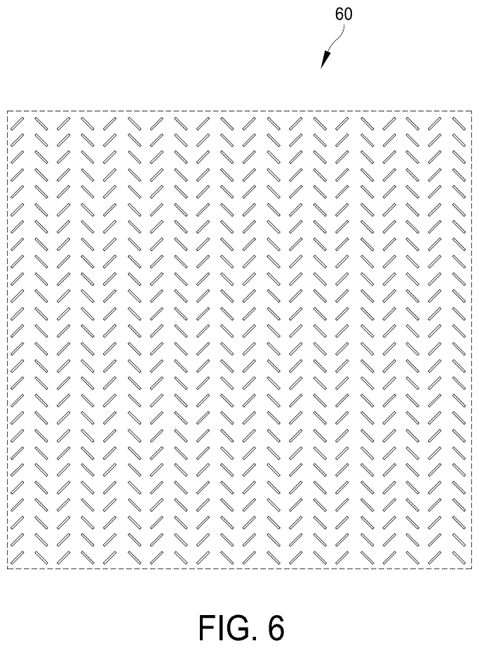

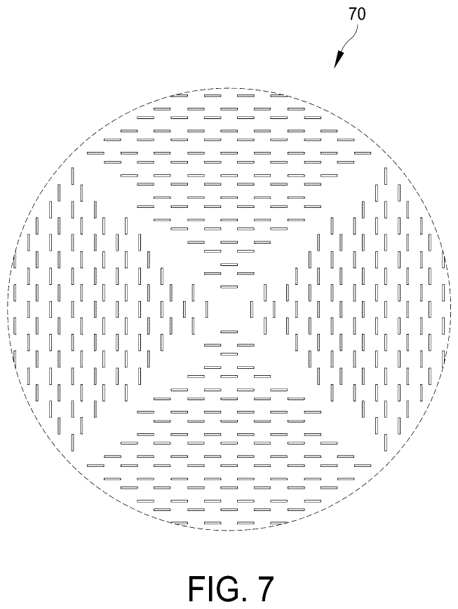

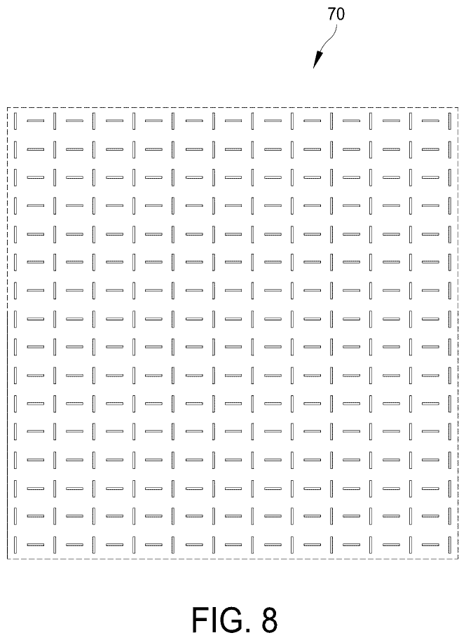

An exemplary method of manufacturing an embodiment of the pre-shaped, meshed ADM graft, and the packaged ADM graft product, may include some or all of the following steps: initiate with providing a portion of full-thickness donor-derived skin. Next, the epidermis layer, basement membrane layer and the fat layer adjacent to the dermis may be removed, and the dermal tissue may be decellularized according to a well-known or a proprietary decellularization process, resulting in the Acellular Dermal Matrix (ADM). The ADM may then be shaped and/or cut into a pre-defined shape, such as the pelvic organ prolapse repair implant for an anterior approach procedure or the pelvic organ prolapse repair implant for a posterior approach procedure or another appropriate shape, as necessary for an associated or pre-determined/assigned surgical procedure. The shaping may be accomplished using any appropriate scoring tool or another appropriate shaping tool, and the graft may be cut out with the cutting tool. As shown in FIGS. 7 and 8, a die system may be configured for cutting the pelvic organ prolapse repair implant for a posterior approach procedure as shown in FIGS. 2 and 6.

The ADM may also be meshed/fenestrated in the desired locations and/or with the desired mesh pattern (e.g., 1:1 graft:space ratio, 2:1 graft:space ratio) using any appropriate skin mesher or fenestrating devices. The meshing or fenestrating process may occur before or after the ADM is shaped into the pre-defined shape. The resulting pre-shaped, meshed ADM graft may then be verified for its thickness to specification (e.g., 1 mm-2 mm) using a thickness gauge, and one or more antimicrobial agents may be added to the pre-shaped, meshed ADM graft to aid in post-surgical infection prevention. The graft may then be packaged between opposing pieces of backing material within sterile water inside a self-sealing medical sterilization pouch and/or a peelable pouch such as, for example, a Kapak peel-pouch, forming the pre-shaped, meshed ADM graft product. The packaged ADM graft product may be irradiated to SAL 10⁻⁶. After irradiation, the packaged, pre-shaped, meshed ADM graft product may be stored up to two years before it is used in a surgical procedure.

The method of manufacturing the packaged, pre-shaped, meshed ADM graft product provides a repeatable process for manufacturing the pre-shaped, meshed ADM graft formed from full-thickness donor-derived skin such that surgeons may rely on the time-saving graft product in reconstructive surgical procedures to provide a graft solution that has the robust physical properties required of surgical skin grafts (as opposed to burn skin grafts), promotes healing in the form of effective drainage from the surgical site, and promotes integration of the graft into the patient's body.

With reference to FIGS. 9 and 10, and in various embodiments, acellular dermal matrix (ADM) graft product 90, 100 may configured for a pelvic organ prolapse repair with additional features. These may include, but are not limited to, an ADM graft 90, 100 derived from full-thickness skin, the ADM graft 90, 100 having a pre-formed shape with mesh pattern 120 formed in central mesh region 115. The mesh pattern 120 may include uniform meshing contained in the central mesh region 115. A suture border region 125A is provided surrounding at least a portion of the central mesh region 115. FIG. 9 illustrates a pre-defined shape of meshed ADM graft 90 configured for an anterior approach procedure of the pelvic organ prolapse repair. FIG. 10 illustrates a pre-defined shape of meshed ADM graft 100 configured for a posterior approach procedure of the pelvic organ prolapse repair. In various embodiments, the ADM graft has a plurality of suture punch points 135 or suture slits placed in the suture border region between the central mesh region 115 and a perimeter edge 125 of the ADM graft product. In at least one embodiment, a plurality of sutures 145 may be pre-placed in the suture border region between the central mesh region 115 and the perimeter edge 125 of the ADM graft product 90, 100.

In another embodiment, there may be provided an acellular dermal matrix (ADM) graft die. The die may have a pre-formed shape configured to create an ADM graft derived from full-thickness skin. In at least one embodiment, the pre-formed shape includes a mesh pattern formed therein. In at one embodiment embodiment, the pre-formed shape is configured for an anterior approach procedure of the pelvic organ prolapse repair. In at least one other embodiment, the pre-defined shape is configured for an posterior approach procedure of the pelvic organ prolapse repair.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

1. (canceled)

2. (canceled)

3. (canceled)

4. (canceled)

5. (canceled)

6. (canceled)

7. A pre-shaped, meshed acellular dermal matrix (ADM) graft configured for a pelvic organ prolapse repair and stored as a packaged graft product prepared by a process comprising the steps of:
providing a portion of ADM tissue having a thickness between 1 mm and 2 mm;
fenestrating the portion of the ADM tissue in a mesh pattern extending over a central portion of the ADM tissue;
scoring the portion of the ADM tissue into a pre-defined shape to form the pre-shaped, meshed ADM graft in the configuration for the pelvic organ prolapse repair;
verifying the thickness of the pre-shaped, meshed ADM graft;
packaging the pre-shaped, meshed ADM graft in a medical sterilization pouch; and
irradiating the pre-shaped, meshed ADM graft within the medical sterilization pouch to a sterility assurance level of 10⁻⁶ to form the packaged graft product.

8. The pre-shaped, meshed ADM graft stored as the packaged graft product prepared by the process of claim 7, wherein the pre-defined shape is configured for an anterior approach procedure of the pelvic organ prolapse repair.

9. The pre-shaped, meshed ADM graft stored as the packaged graft product prepared by the process of claim 7, wherein the pre-defined shape is configured for a posterior approach procedure of the pelvic organ prolapse repair.

10. The pre-shaped, meshed ADM graft stored as the packaged graft product prepared by the process of claim 7, the process further comprising:
prior to the scoring, adding one or more antimicrobial agents to the portion of the ADM tissue.

11. The pre-shaped, meshed ADM graft stored as the packaged graft product prepared by the process of claim 7, wherein the mesh pattern has an ADM tissue:space ratio of 1:1.

12. The pre-shaped, meshed ADM graft stored as the packaged graft product prepared by the process of claim 7, wherein the packaged graft product has a shelf-life of two years.

13. The pre-shaped, meshed ADM graft stored as the packaged graft product prepared by the process of claim 7, wherein the ADM graft has a central mesh region, uniform meshing contained in the central mesh region, and a suture border region surrounding at least a portion of the central mesh region.

14. The pre-shaped, meshed ADM graft stored as the packaged graft product prepared by the process of claim 7, wherein the ADM graft has a central mesh region, uniform meshing contained in the central mesh region, and a plurality of suture punch points or suture slits placed in a non-meshed region between the central mesh region and a perimeter edge of the ADM graft product.

15. The pre-shaped, meshed ADM graft stored as the packaged graft product prepared by the process of claim 7, wherein the ADM graft has a perimeter with a series of radii edges having a rounded configuration to provide decreased stress so as to avoid tearing of the ADM graft.

16. An acellular dermal matrix (ADM) graft product configured for a pelvic organ prolapse repair, comprising:
an ADM graft derived from full-thickness skin, the ADM graft having a pre-formed shape with a mesh pattern formed therein; and
a medical sterilization pouch sealed about the ADM graft, wherein when the medical sterilization pouch and the ADM graft are irradiated to a sterility assurance level of 10⁻⁶, the ADM graft product has a shelf-life of two years.

17. The ADM graft product of claim 16, wherein the pre-defined shape is configured for an anterior approach procedure of the pelvic organ prolapse repair.

18. The ADM graft product of claim 16, wherein the pre-defined shape is configured for a posterior approach procedure of the pelvic organ prolapse repair.

19. The ADM graft product of claim 16, wherein the mesh pattern extends across a central portion of the pre-formed shape, and wherein the mesh pattern has a material:space ratio of 1:1.

20. The ADM graft product of claim 16, wherein the ADM graft has a thickness between 1 mm and 2 mm.

21. The ADM graft product of claim 16, wherein the ADM graft has a central mesh region, uniform meshing contained in the central mesh region, and a suture border region surrounding at least a portion of the central mesh region.

22. The ADM graft product of claim 16, wherein the ADM graft has a central mesh region, uniform meshing contained in the central mesh region, and a plurality of suture punch points or suture slits placed in a non-meshed region between the central mesh region and a perimeter edge of the ADM graft product.

23. The ADM graft product of claim 16, wherein the ADM graft has a perimeter with a series of radii edges having a rounded configuration to provide decreased stress so as to avoid tearing of the ADM graft.

24. An acellular dermal matrix (ADM) graft product configured for a pelvic organ prolapse repair, comprising:
an ADM graft derived from full-thickness skin, the ADM graft having a pre-formed shape with a mesh pattern formed in a central mesh region, wherein the mesh pattern is uniform meshing contained in the central mesh region, and a suture border region surrounding at least a portion of the central mesh region.

25. The ADM graft product of claim 24, wherein the pre-defined shape is configured for an anterior approach procedure of the pelvic organ prolapse repair.

26. The ADM graft product of claim 24, wherein the pre-defined shape is configured for a posterior approach procedure of the pelvic organ prolapse repair.

27. The ADM graft product of claim 24, wherein the ADM graft has a plurality of suture punch points or suture slits placed in the suture border region between the central mesh region and a perimeter edge of the ADM graft product.

28. The ADM graft product of claim 24, wherein the ADM graft has a plurality of sutures pre-placed in the suture border region between the central mesh region and a perimeter edge of the ADM graft product.
